(19) United States
(12) Patent Application Publication
(10) Pub. No.: US 2017/0119538 A1
(43) Pub. Date: May 4, 2017

(54) EXPANDABLE SPINAL FUSION DEVICE

(71) Applicant: Atlas Spine, Inc., Jupiter, FL (US)

(72) Inventor: Matthew Glen Baynham, Jupiter, FL (US)

(21) Appl. No.: 15/261,322

(22) Filed: Sep. 9, 2016

Related U.S. Application Data
(60) Provisional application No. 62/216,656, filed on Sep. 10, 2015.

(51) Int. Cl. A61F 2/44

(52) U.S. Cl. CPC A61F 2/447; A61F 2002/30579

(57) ABSTRACT

An implant formed from an expandable spinal device which incorporates a main body, a flexible front body, a push arm, and a drive screw with the intention of expanding the footprint (surface area and bone grafting volume) of the device after insertion.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 62/216,656, entitled “EXPANDABLE SPINAL FUSION DEVICE”, filed Sep. 10, 2015. The contents of the above referenced application are incorporated herein by reference in its entirety.

### FIELD OF INVENTION

This invention relates to the field of orthopedic surgery and, more particularly, to implants placed between vertebrae in the spine.

### BACKGROUND OF THE INVENTION

Spinal stabilization is known to alleviate chronic back pain caused by displaced disk material or excessive movement of individual vertebrae. Conventional stabilization techniques include fusing two or more vertebrae together to immobilize the area of excessive movement. In one technique, vertebral disk material which separates the vertebrae is removed and bone graft material is inserted in the space for interbody fusion. In addition to or, in place of, the bone graft material, a spinal implant may be inserted in the intervertebral space.

The conventional surgical approach for stabilization has been posterior for ease of access to the spine and to avoid interfering with internal organs and tissue. Usually the implant site is prepared to maintain natural lordosis and to accept a certain sized implant within certain pressure limits. This requires considerable time and skill by the surgeon.

Disclosed is an implant forming an expandable spinal device which incorporates a main body, a flexible front body, a push arm, and a drive screw with the intention of expanding the footprint (surface area and bone grafting volume) of the device after insertion.

It is an objective of the instant invention to teach a lateral surgical approach for the placement of an intervertebral spacer implant for interbody fusion allowing the implant to be inserted through a small incision and increased in size in situ.

Other objectives and advantages of this invention will become apparent from the following description taken in conjunction with any accompanying drawings; wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is an isometric view of the implant in place in the spine.

FIG. 2 is a side view of the implant between two vertebral bodies.

FIG. 3 is a side view of the implant in the expanded state between two vertebral bodies.

FIG. 4 is a top view of the implant in position on the spine.

FIG. 5 is a top view of the implant in the expanded state in position on the spine.

FIG. 6 is a prospective view of the implant.

FIG. 7 is a prospective view of the implant in the expanded state.

FIG. 8 is a top view of the implant.

FIG. 9 is a top view of the implant in the expanded state.

FIG. 10 is a side view of the implant.

FIG. 11 is a side view of the implant.

FIG. 12 is a back view of the implant.

FIG. 13 is a front view of the implant.

FIG. 14 is an exploded view of the implant.

FIG. 15 is an exploded isometric view of the implant.

FIG. 16 is an exploded isometric view of the implant.

FIG. 17 is an exploded isometric view of the implant.

FIG. 18 is an exploded isometric view of the implant.

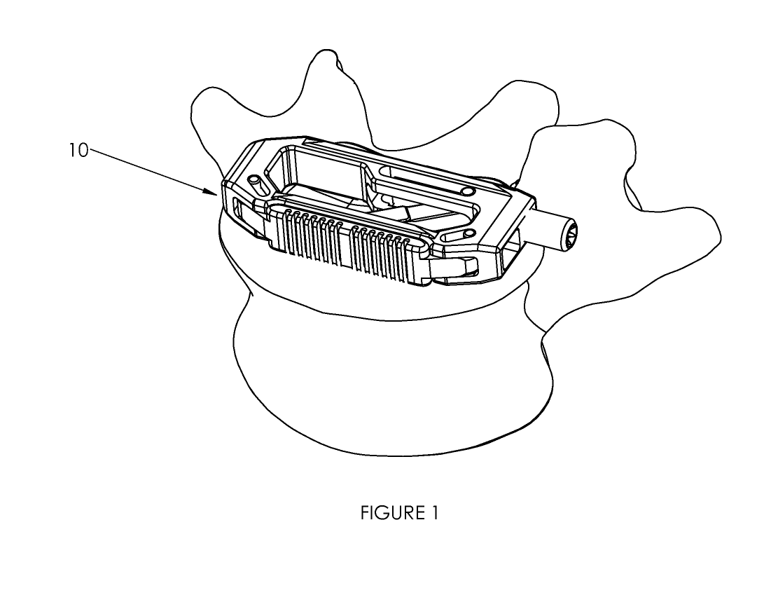

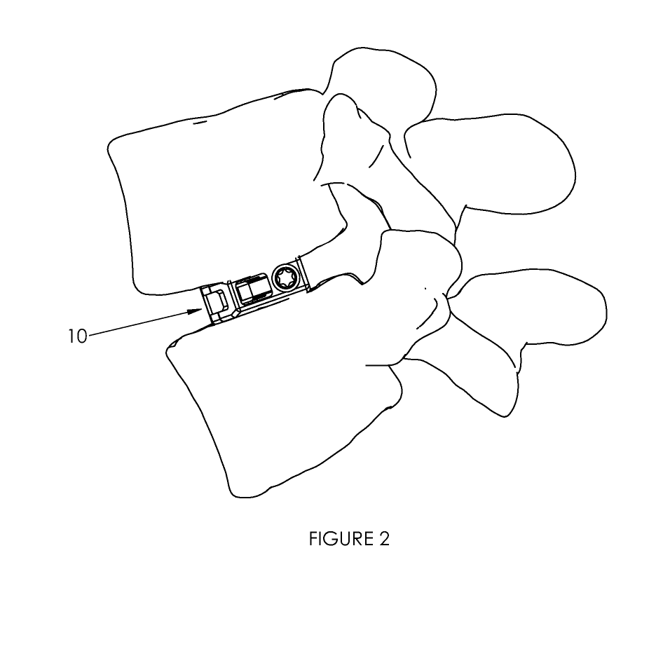

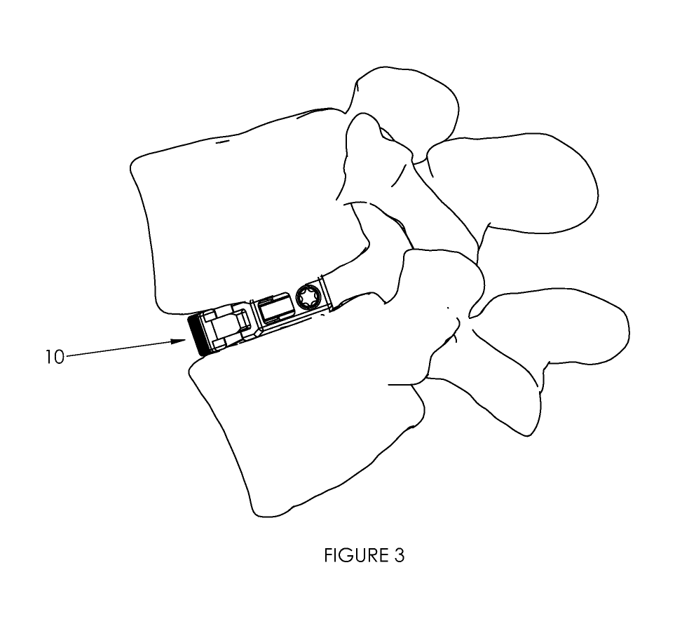

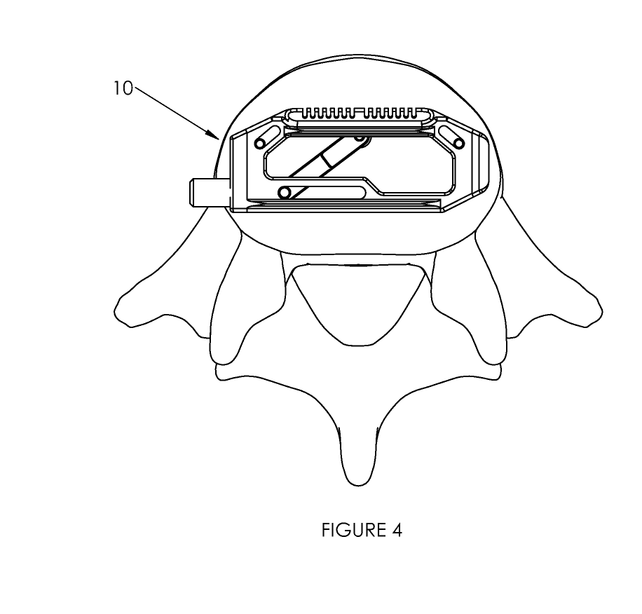

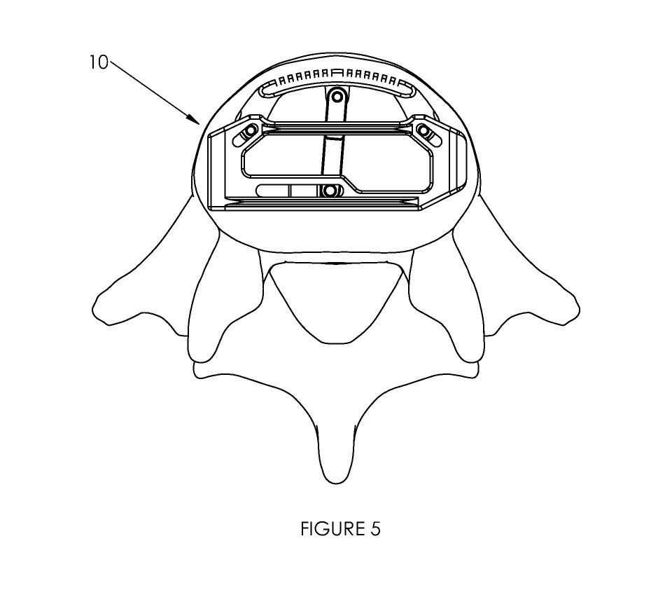

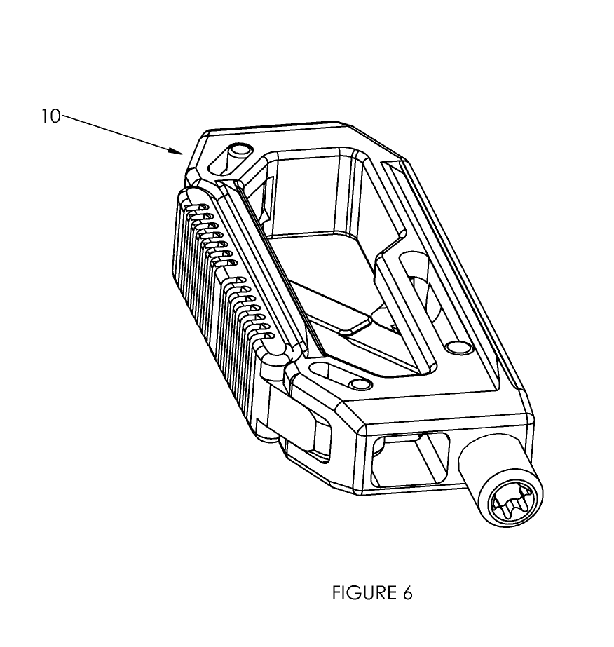

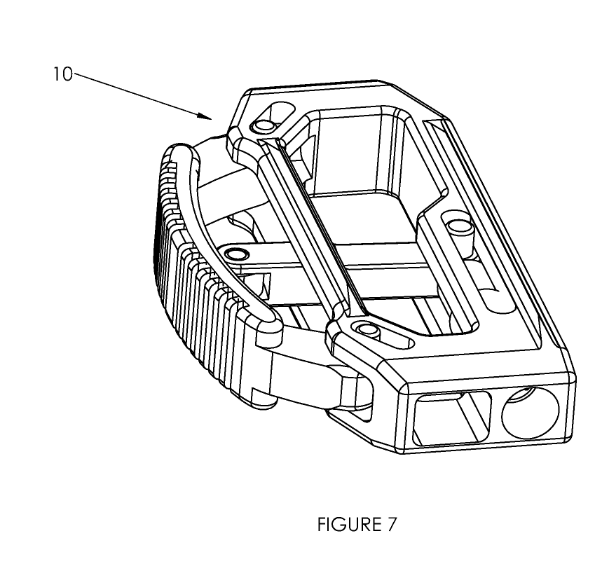

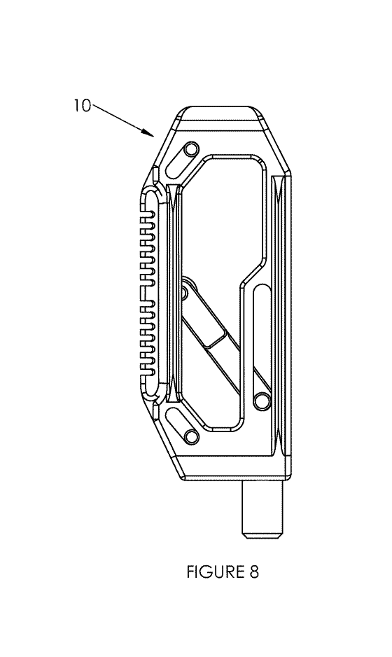

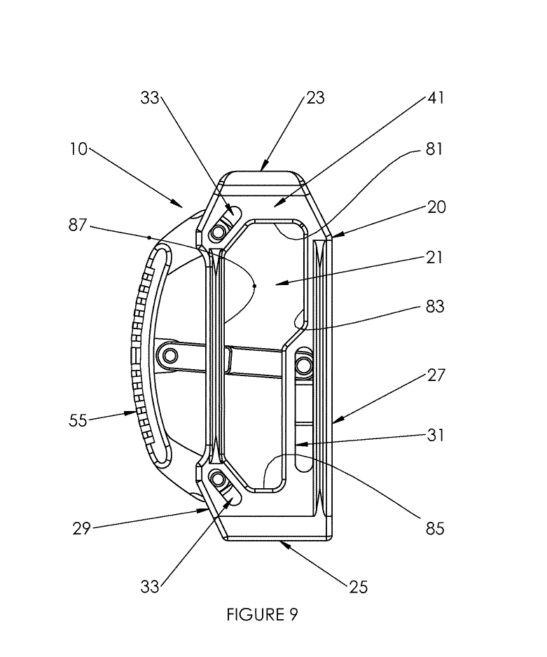

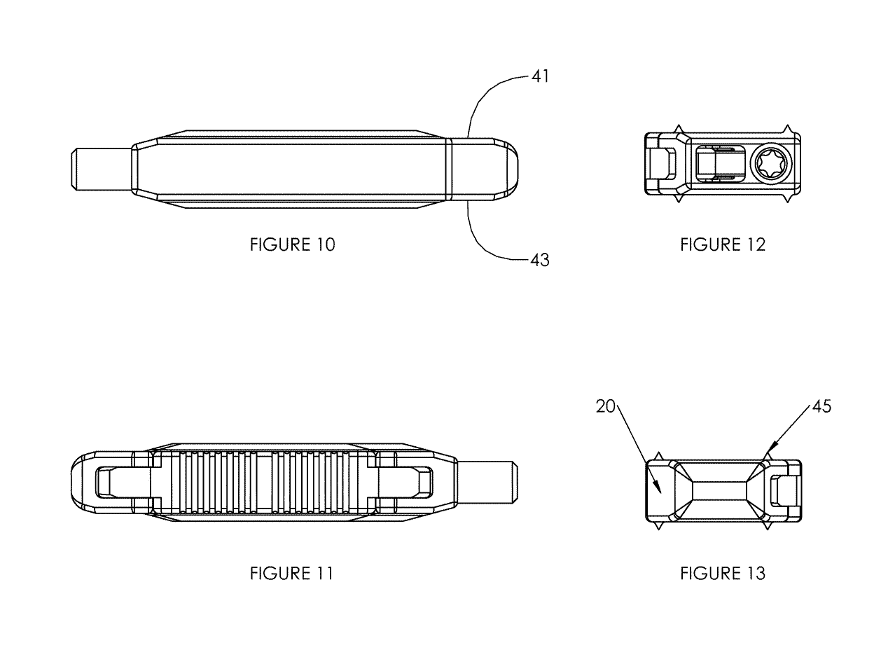

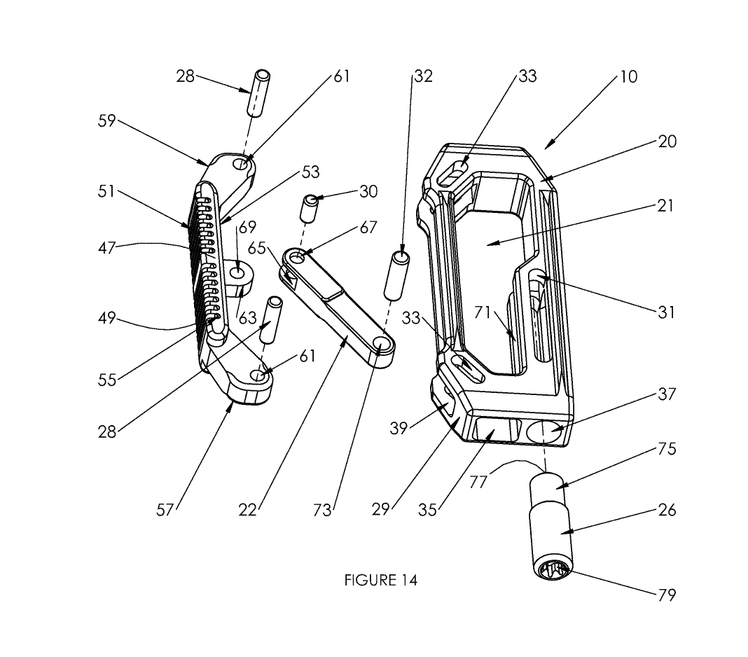

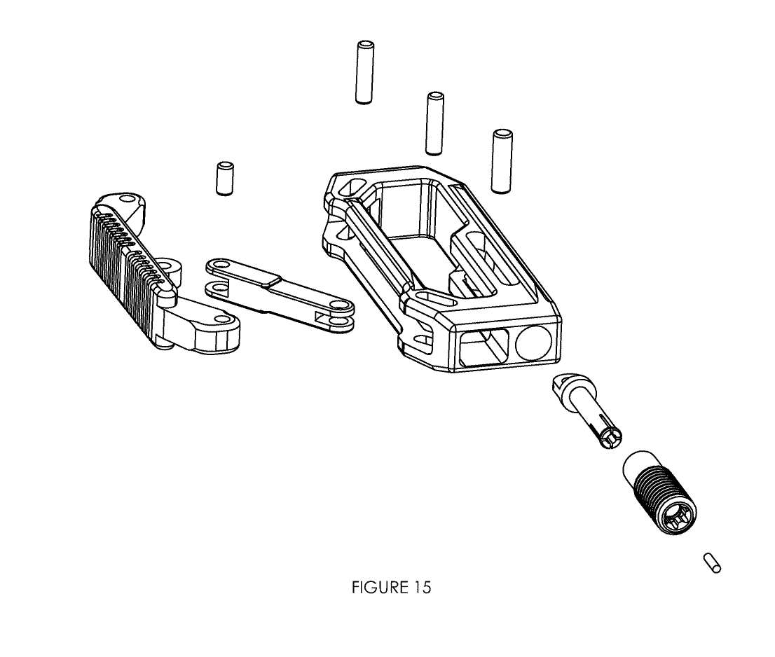

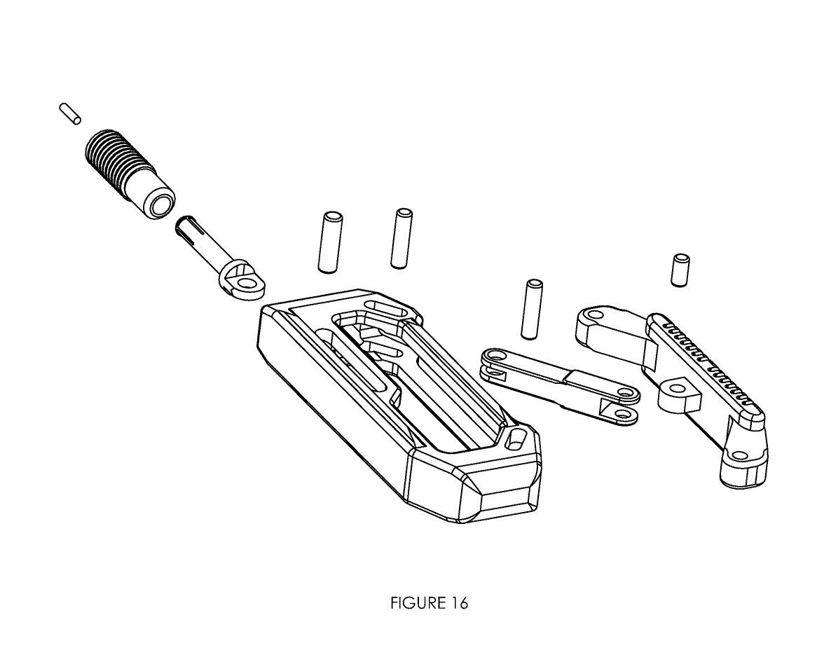

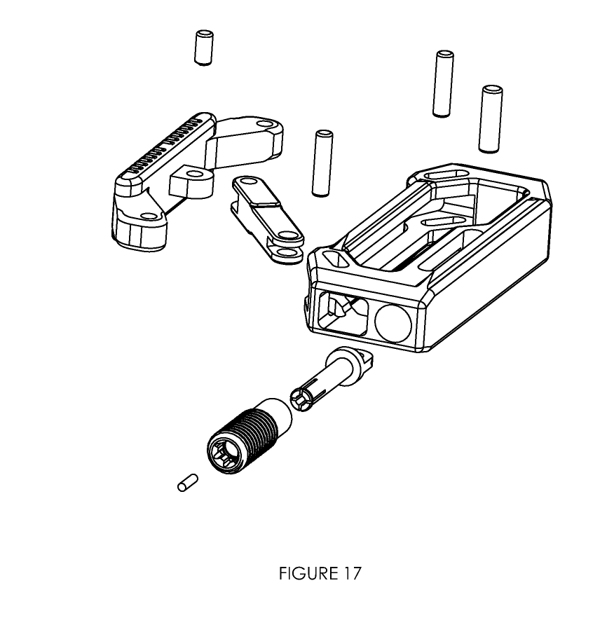

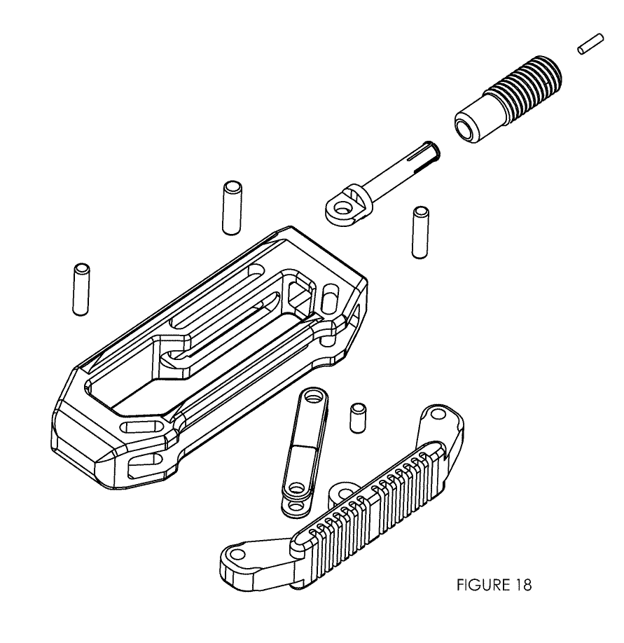

### DETAILED DESCRIPTION OF THE INVENTION

The implant 10 is inserted in the intervertebral space to replace damaged, missing, or excised disk material. The implant 10 is placed between adjacent vertebrae which are forced apart to a desired spacing.

Referring now in general to the figures, depicted is the implant 10 of the instant invention which consists of a main body 20, a push arm 22, a flexible body 24, a drive screw 26, flexible body guide pins 28, a push arm pivot pin 30, and a push arm guide pin 32.

The main body 20 has an aperture 21 that extends through it to create and open architecture. The aperture is defined by at least four interior walls 81, 83, 85, and 87. The main body 20 has a distal end 23, a proximal end 25, a side wall 27, and a side wall 29 formed to make a reasonably rectangular shape. The implant 10 has a slot 31 which extends through the main body 20 as a travel limit for the push arm guide pin 32. The main body 20 also has slots 33 configured to act as travel limiters for the flexible body guide pins 28. The main body 20 incorporates an opening 35 at the proximal end which extends into the main cavity of 20. The main body 20 has a threaded hole 37 at its proximal end to receive the drive screw 26. The main body 20 has a slot 39 which extends through its side wall 29 to receive push arm 22 and flexible body 24. The main body 20 has a top surface 41 and a bottom surface 43. The main body 20 incorporates extended portions 45 as stabilizers (anchors) to maintain position during the expansion maneuver.

The implant 10 can be made of conventional materials commonly used in surgical implants, such as stainless steel and its many different alloys, titanium, or any other material with the requisite strength and biologically inert properties. Polymeric materials with adequate strength and biological properties may also be used in the construction of the main body 20, the push arm 22, the flexible body 24, the drive screw 26, the flexible body guide pins 28, the push arm pivot pin 30, and the push arm guide pin 32.

The flexible body 24 has a top surface 47, a bottom surface 49, an exterior side wall 51, and an interior side wall 53. The flexible body 24 incorporates an arrangement of slots 55 which extend from the top surface 47 through the bottom surface 49 in a pattern along the exterior wall 51 to allow for a more flexible member. The flexible body 24 has extensions 57 and 59 that correspond with slot 39 in the main body 20. The extensions 57 and 59 of the flexible body 24 incorporate thru holes 61 to receive the flexible body guide pins 28 which travel along slot 33 in the main body 20 in the assembled state. The flexible body 24 has an extension 63 which communicates with slot 65 in the push arm 22.

The push arm 22 and the flexible body 24 are joined in assembly by the push arm pivot pin 30 which goes through a thru hole 67 in the push arm 22 and a thru hole 69 in the flexible body 24. The push arm 22 rides through slot 39 and slot 71 of the main body 20.

The push arm guide pin 32 goes through slot 31 and thru hole 73 in the push arm 22 in the assembled state. The push arm guide pin 32 travels in a restricted fashion in slot 31 of the main body 20 as the drive screw 26 is advanced. The drive screw 26 has a threaded portion 75 which communicates with the threaded opening of 37 of the main body 20. The drive screw 26 has a distal end 77 which communicates with the push arm 22 to drive the expansion feature. The drive screw has a receiving end 79 to receive an appropriate screwdriver (not shown).

As the drive screw 26 is advanced, the push arm 22 changes its angle as it travels towards the distal end of slot 31. With the push arm 22 rigidly connected to the flexible body 24, this change in angle applies a force that bends the flexible body 24 and translates it along the flexible body guide pins 28 until they terminate their slot 33.

What is claimed is:

1. The product comprising any feature described, either individually or in combination with any feature, in any configuration.

2. The process comprising any process described, in any order, using any modality, either individually or in combination with any feature, in any configuration.
